(19) United States
(12) Patent Application Publication
(10) Pub. No.: US 2023/0302381 A1
(43) Pub. Date: Sep. 28, 2023

(54) METAL-AFFINITY EXTRACTION OF HOST CELL DNA

(71) Applicant: Sartorius BIA Separations d.o.o., Ajdovscina (SI)

(72) Inventors: Peter S. GAGNON, Las Vegas, NV (US); Sara Drmota PREBIL, Ajdovscina (SI); Maja LESKOVEC, Ajdovscina (SI); Ales STRANCAR, Ajdovscina (SI)

(21) Appl. No.: 18/021,421

(22) PCT Filed: Aug. 18, 2021
(86) PCT No.: PCT/EP2021/072951

§ 371 Date: Feb. 15, 2023

(30) Foreign Application Priority Data
Aug. 18, 2020 (EP) 20191587.3

(51) Int. Cl. B01D 15/38; B01D 15/18; B01D 15/20; C07K 14/075; B01D 15/36

(52) U.S. Cl. CPC B01D 15/3828; B01D 15/1864; B01D 15/203; C07K 14/075; B01D 15/363

(57) ABSTRACT

A method for removal of host cell DNA from a sample containing a species of desired protein, virus, or extracellular vesicle comprising the steps of:
Loading a substrate bearing an anionic metal affinity ligand with a metal ion,
Equilibrating the substrate with a buffer having a pH in the range of pH 6 to pH 10, and a salt concentration in a concentration range up to 1 M which salt is not forming a chemical complex with the anionic metal affinity ligand,
Contacting the sample with the metal-loaded anionic metal affinity substrate,
Separating the substrate from the sample, wherein the sample has reduced content of contaminating DNA.

The present invention pertains to method for removal of host cell DNA from a sample containing a desired species of a protein, a virus, or an extracellular vesicle.

### BACKGROUND

Host cell-derived DNA is a ubiquitous contaminant of all biologics produced by cell culture. Regulatory agencies require that host DNA contamination of biological therapeutics be reduced to extremely low levels. They do so to minimize the probability of inadvertent transmission of pathogenic-viral or oncogenic DNA sequences to patients receiving therapy.

Although host cell DNA is referred to as if it exists in cell cultures as an independent contaminant class, it is always strongly associated with proteins, mostly in well-defined compound structures. Host cell DNA in cell culture harvests is the remnant of the chromosomal mass of the cells that were used to produce the biological therapeutic of interest. This mass is referred to as chromatin. Chromatin principally includes chromosomal DNA and the histone proteins that compact the DNA and regulate transcription in the nucleus of the cell. In cell culture harvests, chromatin is degraded into linear arrays of one to about 30 nucleosomes, ranging in size from about 12-400 nm, and smaller histone-associated DNA fragments ranging from about 2-12 nm. A subset of the fragments form compound assemblages with nucleosomal arrays.

Chromatin contamination of cell culture harvests and cell lysates is also important because chromatin interacts non-specifically with all known purification methods and media [1-3]. It is documented to reduce capacity, inflate contamination by host proteins, and inflate aggregate content but it also causes excessive levels of DNA to persist across multistep purification processes that are logically expected to remove it.

Host DNA levels can be adequately reduced in some cases during the course of multistep chromatographic purification but, in all cases, DNA reduction is enhanced if a portion of the chromatin load is removed before chromatographic purification commences. Many methods of advance chromatin removal have been described in the field of IgG purification, including co-precipitation with positively charged particles, flocculation with positively charged polymers, flocculation with positively charged organics, and removal with positively charged depth filtration media [1-4]. DNA binds to positively charged anion exchange chromatography columns but they are impractical for bulk DNA removal because they become fouled and clogged by the large amounts of chromatin in cell culture harvests and cell lysates.

Flocculation with negatively charged organic reagents (fatty acids) particularly targets histone proteins but co-precipitates the host DNA associated with them. Combinations of fatty acids with positively charged flocculating agents are more effective than either alone. Their effectivity is enhanced further when both are combined with allantoin, which particularly tends to remove large species such as high molecular weight aggregates [1,2].

Extraction of DNA from some cell culture products is simpler than others because the properties of different product classes overlap to differing degrees with DNA. The chemistry of IgG monoclonal antibodies is fundamentally distinct from DNA. This makes it practical to apply a wide variety of chemical methods to selectively remove DNA from antibody preparations. Most such treatments have no interaction with IgG, which therefore remains in solution and can be recovered with high yield.

DNA reduction from preparations of viruses and extracellular vesicles is more challenging because they share several chemical similarities, including a net negative charge conferred at least in part by the presence of phosphate groups. This disqualifies DNA extraction methods that exploit positive charges since the positive charges remove the viruses and vesicles along with the DNA. It also implies that methods exploiting affinity for phosphate residues will be compromised. Flocculation with fatty acids is also disqualified from use with lipid enveloped viruses and vesicles because fatty acids destabilize their lipid membranes. Allantoin is likewise disqualified because it indiscriminately co-precipitates large species. Viruses and vesicles occupy the same range of sizes as chromatin in cell harvests so allantoin removes them along with the chromatin.

The absence of methods for DNA removal in the fields of virus and vesicle preparation has led to a reliance on DNA reduction by enzymatic lysis. This approach is partially successful but cannot remove all the host cell DNA because it is protected by its strong associations with histone proteins.

The method of immobilized metal affinity chromatography (IMAC) is known for purification proteins, viruses, exosomes, and DNA into which a metal-binding site in the form of a polyhistidyl (his-tag) has been recombinantly introduced. Such columns are most often eluted with imidazole; less often by reducing pH. Purification of RNA is known using IMAC columns employing iminodiacetic acid loaded with copper, nickel, zinc, and cobalt (strongest to weakest) [5]. RNA binds these metals through formation of coordination bonds with its nitrogen bases, with purine bases binding more strongly than pyrimidines.

DNA shows low affinity for IMAC because its nitrogen bases are sterically inaccessible as a result of being involved in base pairing between strands. This causes most of the DNA to pass through the column. The inability of IMAC columns to bind DNA can be overcome by thermal or alkaline dissociation of DNA into individual strands so that its nitrogen bases become sterically accessible to the metal ions on the surface of the IMAC media [6].

The use of anion exchange chromatography with elution by salt gradient for separation of empty and full adeno-associated virus capsids is known [7-9].

### SUMMARY OF THE INVENTION

In one aspect the method of the invention may be used for removal of host cell DNA from a sample containing a desired species of a protein, a virus, or an extracellular vesicle. The method of the invention comprises the steps of:
Loading a substrate bearing an anionic metal affinity ligand with a metal ion,
Equilibrating the substrate with a buffer having a pH in the range of pH 4 to pH 10, and a salt concentration in a concentration range up to 1 M which salt is not forming a chemical complex with the metal-loaded anionic metal affinity substrate ligand,
Contacting the sample with the metal-loaded anionic metal affinity substrate,
Separating the substrate from the sample, wherein the sample has reduced content of host cell DNA.

Equilibrating the substrate with an equilibration buffer is typically performed by adjusting the buffer conditions to a combination of pH and salt conditions that prevents binding of a virus or extracellular vesicle but permits binding of contaminating DNA. The respective conditions to be selected can be easily determined by the person skilled in the art. As an indication, the pH can be adjusted in a range of pH 6 to pH 10 and the salt concentration may be up to 1 M.

In the attempt to relief legibility of the description, the technically correct but rather ponderous expression “salt which is not forming a chemical complex with the metal-loaded anionic metal affinity substrate” has been replaced by an informal term “non-chelating salt”. It is believed that the skilled person understands passages where the more informal term “non-chelating salt” is used the more technical expression “salt which is not forming a chemical complex with the metal-loaded anionic metal affinity substrate” shall be meant. Therefore, in the following, for the sake of simplicity the expression “salt which is not forming a chemical complex with the metal-loaded anionic metal affinity substrate” is also referred to “non-chelating salt”.

It is understood that the terms “host DNA” or “contaminating DNA” is meant to be DNA which is not encapsulated into particles or adsorbed on particles such as viruses, virus-like particles, capsids, vesicles, exosomes, liposomes and the like.

In a second aspect the present invention pertains to a two-step method that is effective for extraction of chromatin from biological products produced by cell culture but is especially distinctive in its ability to selectively remove host DNA from preparations of virus particles and extracellular vesicles. The first step consists of treating a sample containing a desired protein, virus, or extracellular vesicle containing excess host cell DNA with an anionic metal affinity substrate loaded with a metal ion. The second step consists of processing the metal-affinity treated sample by anion exchange chromatography.

In one embodiment of the method of the invention the anionic metal affinity ligand may be selected from the group consisting of amino-dicarboxylic acids and amino tricarboxylic acids.

In a further embodiment of the method of the invention the anionic metal affinity ligand can be iminodiacetic acid (IDA) or nitriloacetic acid (NTA).

In yet another embodiment of the method of the invention the substrate bearing an anionic metal affinity ligand can be in the form of particles, nanofilaments, porous membranes, monoliths, hydrogels, depth filtration media, soluble polymer media. In particular, the substrate bearing an anionic metal affinity ligand can be in the form of a flow-through chromatography device.

In yet a further embodiment of the method of the invention equilibrating the substrate and/or the sample may be performed by means of a buffer having a pH in the range of pH 7.0 to 9.5, 7.0 to 9.0, 7.5 to 9.0 or 8.0 to 9.0.

In still another embodiment of the method of the invention equilibrating the substrate and/or the sample can be performed by means of a buffer having a salt concentration in the range of up to 1 M, or 50 mM to 750 mM, or 100 mM to 500 mM, or 125 mM to 250 mM.

In one embodiment of the method of the invention the buffer can be adjusted by means of a salt which is not forming a chemical complex with the metal-loaded anionic metal affinity substrate selected from the group consisting of an inorganic salt, such as sodium chloride, or potassium chloride, or sodium acetate, or potassium acetate; an organic salt, such as arginine-HCl, lysine-HCl, or a salt based on an imidazolium, histidyl, or histaminyl cation; and a chaotropic salt such as comprising a guanidinium cation or a thiocyanate anion, or both; and combinations thereof. The skilled person is readily able to adjust the salt conditions in a manner that prevent binding of virus or extracellular vesicles but permit binding of DNA.

In another embodiment of the method of the invention the metal-loaded anionic metal affinity substrate can be loaded with metal ions having at least two positive charges, preferably selected from the group consisting of calcium, magnesium, copper, iron, manganese, zinc, barium, nickel, cobalt, and combinations thereof.

In a further embodiment of the method of the invention the sample of viruses and extracellular vesicles may comprise cell harvests, cell lysates or entities selected from the group consisting of non-lipid-enveloped protein capsid virus particles, such as AAV capsids; lipid-enveloped virus or virus-like particles, such as an influenza virus or a corona virus; bacteriophages, extracellular vesicles, such as are exosomes; and combinations thereof. In particular the AAV capsid is selected from the group consisting of AAV serotypes AAV1, AAV2, AAV3, AAV4, AAV5, AAV6, AAV7, AAV8, AAV9, AAV10, AAV11, a recombinant hybrid serotype like AAV2/8, or AAV2/9, a synthetic recombinant serotype and combinations thereof.

In one embodiment of the method of the invention the sample can be processed by biological affinity chromatography, cation exchange after metal affinity, hydrophobic interaction chromatography, and/or tangential flow filtration before or after the method for removal of contaminating DNA. Biological affinity chromatography means a chromatography utilizing ligands having an affinity to molecules or molecular structures and are typically of proteinaceous nature such as antibodies, antibody fragments, e.g. Fc or Fab fragments; lectins, protein A. But also other ligands are known to the person skilled in the art, e.g. biotin/avidin.

In a further embodiment of the method of the invention the tangential flow filtration may be using a membrane with pore size cutoffs in the range of up to 1 MDa, in particular 200 kDa to 700 kDa.

In a preferred embodiment, the sample containing the desired species is a cell harvest or cell lysate. In this embodiment, the method of the present invention is applied directly after harvest or lysis to a clear or clarified sample. Such clarification may include a filtration step. In this embodiment, no ion-exchange chromatography step is applied prior to the method of the invention. Preferably, no chromatography step is applied prior to the method of the invention. In a preferred embodiment, the method of the invention uses pH and salt concentration that avoids binding of the desired species to the metal-loaded anionic metal affinity substrate.

In still a further embodiment of the method of the invention the sample having a reduced content of DNA is processed by anion exchange chromatography to further reduce the level of contaminating DNA.

Another aspect of the invention is also the use of a substrate bearing an anionic metal affinity ligand in a method for removal of contaminating DNA from a sample of viruses and extracellular vesicles under alkaline conditions.

The utility of the method derives from a series of unexpected discoveries. The first is that the viruses and vesicles have a natural tendency to bind substrates bearing anionic metal affinity ligands complexed with certain metals even when they lack genetic modifications, such as His-tags, to mediate metal affinity binding. Their natural metal affinity results in their partial binding to metal affinity substrates, which results in loss of the bound product. It has been discovered that such binding can be reduced with alkaline pH. This is surprising since metal affinity methods are known where acidic pH elutes IgG and His-tag proteins but not the opposite situation where increasing pH is able to cause elution or prevent binding. The expectation is that increasing pH should maintain or increase binding.

More surprising has been the discovery of an inverse relationship between pH and non-chelating salt concentration, where increasing amounts of non-chelating salts overcome stronger binding of viruses and extracellular vesicles at neutral and acidic values. This was unexpected because it is known throughout the field of immobilized metal affinity chromatography (IMAC) that salts which are not forming a chemical complex with the metal-loaded anionic metal affinity substrate (non-chelating salts) generally do not influence metal affinity binding. No examples are known in the art where proteins bound by metal affinity can be eluted by increasing the concentration of non-chelating salts. That there should be an inverse relationship between pH and salt concentration is even more unexpected.

Yet more surprising has been the discovery that the conditions that weaken binding of viruses and extracellular vesicles permit binding of host cell DNA despite the prior art indicating that DNA binds poorly [5,6]. RNA is known to bind immobilized metals but only copper, nickel, zinc, and cobalt, in order of strongest to weakest, respectively. The present method offers its strongest DNA-binding with iron and manganese but also works with magnesium, calcium, and barium, emphasizing further that the mechanism is distinct from known methods of nucleic acid binding with immobilized metal substrates.

The mechanism by which DNA binding occurs remains unknown. Since the DNA is not denatured into its constituent single strands by heat or sodium hydroxide, it appears that binding cannot occur through its nitrogen bases. This leaves the possibility that DNA binds metal through its phosphate residues. However, this highlights a compromise since the same metals would be expected to bind the phosphate residues on viruses and vesicles. Neither viruses nor vesicles bind under the conditions specified for practicing the method.

These surprising discoveries together create the foundation for a novel approach to removal of host DNA, especially from preparations containing viruses and extracellular vesicles. Experimental results suggest that the method somehow “threads the needle,” with binding conditions that sufficiently reduce metal affinity for proteins, viruses, and extracellular vesicles while conserving the ability of the metal affinity substrate to bind DNA. The DNA-deficient post-treatment sample is subsequently applied to an anion exchanger and eluted by means of a gradient that releases the desired product while still retaining DNA.

In brief, the method consists of a series of steps: a substrate bearing an anionic metal affinity ligand is loaded with a metal ion. The substrate is equilibrated to a combination of pH and salt conditions that prevents binding of a desired protein, virus, or extracellular vesicle but permits binding of DNA. A sample containing a desired protein, virus, or extracellular vesicles contaminated with DNA is equilibrated to the same conditions and contacted with the metal-loaded anionic metal affinity substrate. The substrate is subsequently separated from the sample, leaving the sample deficient in DNA but still containing most of the desired protein, virus, or extracellular vesicles. The metal affinity-treated sample is then processed by anion exchange chromatography to further reduce the level of contaminating DNA.

In one non-limiting example describing application of the method to a preparation containing adeno-associated virus (AAV) and host cell DNA:
A solid phase bearing an anionic metal affinity ligand is loaded with a metal such as iron.
The solid phase is equilibrated to a pH of about 9. This will be recognized as highly unusual in the field of metal affinity chromatography, where sample application is customarily performed at neutral pH.
A cell lysate containing host cell DNA and AAV is equilibrated to a pH of about 9.
The sample is contacted with the metal affinity solid phase. The majority of the AAV does not bind. Host cell DNA is bound.
The anionic metal affinity solid phase is separated from the sample, leaving the DNA bound to the solid phase.
The AAV is fractionated by anion exchange chromatography to further reduce the content of host cell DNA.

In another non-limiting example, the conditions and steps of the previous example are repeated through separation of the anionic metal affinity substrate from the sample. Thereafter:
The sample is processed by tangential flow filtration using membranes with a pore size cutoff rating of 300 kDa to concentrate the AAV and reduce contamination by proteins.
The AAV is fractionated by anion exchange chromatography to further reduce the content of host cell DNA.

In another non-limiting example, the conditions and steps of the first example are repeated through separation of the anionic metal affinity substrate from the sample. Thereafter:
The sample is processed by affinity chromatography.
The AAV is fractionated by anion exchange chromatography to further reduce the content of host cell DNA.

The method of the invention also works with proteins, including antibodies, where it may prove advantageous over known methods that exploit other chemical mechanisms to reduce the content of host cell DNA.

The method of the invention is depicted in FIG. 1. Additional details and variations illustrating the full scope of the method are provided in the following sections.

### BRIEF DESCRIPTION OF THE FIGURES

FIG. 1 depicts a diagram of the method of the invention.

FIG. 2 depicts non-binding of AAV capsids to an anionic metal affinity ligand loaded with magnesium at pH 9.0, while DNA binds strongly and requires sodium hydroxide for elution.

FIG. 3 depicts a comparison of AAV capsid binding to an anionic metal affinity ligand loaded with magnesium in separate experiments at pH 7.0 and pH 9.0. Profiles at 280 nm.

FIG. 4 depicts DNA removal and fractionation of empty and full AAV capsids by a quaternary amine anion exchanger eluted with a salt gradient.

FIG. 5 depicts DNA removal and fractionation of empty and full AAV capsids by a primary amine anion exchanger eluted with a pH gradient.

FIG. 6 depicts size exclusion chromatography of cell culture containing extracellular vesicles, including exosomes.

FIG. 7 depicts size exclusion chromatography of cell culture containing extracellular vesicles after removal of DNA by the method of the invention.

FIG. 8 depicts the size exclusion elution profiles of FIGS. 7 and 8 overlaid to highlight the reduction of contaminants in general, particularly including DNA.

FIG. 9 depicts DNA removal and fractionation of partially purified extracellular vesicles with a quaternary amine anion exchanger eluted with a salt gradient.

FIG. 10 depicts bacteriophage T4 flowing through an iminodiacetic acid monolith loaded with ferric iron.

FIG. 11 depicts secondary removal of DNA and fractionation of bacteriophage T4 by chromatography on a quaternary amine anion exchanger eluted with a salt gradient.

FIG. 12 depicts secondary removal of DNA and fractionation of bacteriophage T4 by chromatography on a primary amine anion exchanger eluted with a salt gradient.

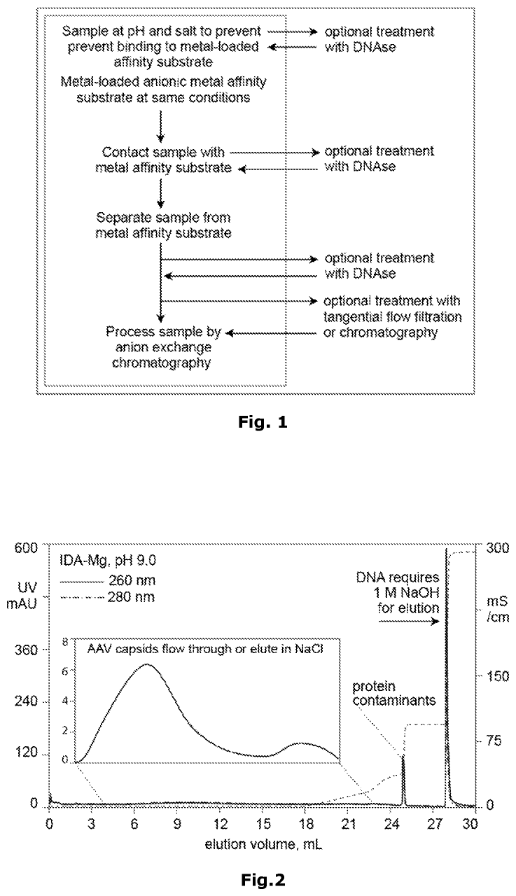

### DETAILED DESCRIPTION OF THE INVENTION

The sample consists of a preparation containing a desired species of proteins, virus particles, or extracellular vesicles produced by cell culture, and also containing host cell-derived DNA. In one embodiment, the sample consists of a cell culture harvest. In one such embodiment, the cell culture harvest contains an antibody. In another such embodiment, the cell culture harvest contains a virus or virus-like particle. In another such embodiment, the cell culture harvest contains extracellular vesicles. In another embodiment, the sample consists of a cell lysate. In another embodiment, the sample consists of a cell culture harvest or cell lysate that has been treated with nuclease enzymes to reduce host cell DNA content. In another embodiment, the sample consists of a partially purified preparation still containing more host cell DNA than is desired or permitted in the final product. In one such embodiment, the sample is a product fraction eluted from a chromatography device. In one such embodiment, the sample is the eluted product from an affinity chromatography column. In another such embodiment, the sample is the eluted product from a size exclusion chromatography column. In another such embodiment, the sample is the eluted product from a hydrophobic interaction chromatography column. In another such embodiment, the sample is the eluted product from a cation exchange chromatography column. In another such embodiment, the sample is the eluted product from an immobilized metal affinity chromatography column. In another such embodiment, the sample is the eluted product from an apatite chromatography column. In another such embodiment, the sample is concentrated and/or diafiltered product from tangential flow filtration.

In one embodiment, the method of the invention is used to process a sample that contains a desired non-lipid-enveloped protein-capsid virus particles contaminated with host cell DNA. AAV has many serotypes. In one embodiment, the desired AAV serotype processed by the method of the invention may be AAV1, or AAV2, or AAV3, or AAV4, or AAV5, or AAV6, or AAV7, or AAV8, or AAV9, or AAV10, or AAV11, or another serotype. In another embodiment, the AAV serotype processed by the method of the invention may be a recombinant hybrid serotype like AAV2/8, or AAV2/9, or another hybrid serotype. In another embodiment, the AAV serotype processed by the method of the invention may be a synthetic recombinant serotype. In any of these embodiments, the anion exchange step may be performed to separate empty capsids from full capsids while further reducing the content of contaminating DNA. In one such embodiment, the anion exchanger is a strong anion exchanger (quaternary amine) eluted with salt. In another such embodiment the anion exchanger is a weak anion exchanger (primary amine) eluted with an ascending pH gradient.

In another embodiment, the sample contains a desired lipid-enveloped virus or virus-like particles contaminated with host cell DNA. In one such embodiment, the anion exchange step may separate non-infective virus particles from infective virus particles. In one such embodiment, the virus is an influenza virus. In another such embodiment, the virus is a corona virus.

In another embodiment, the sample contains a desired bacteriophage contaminated with host cell DNA.

In another embodiment, the method of the invention is used to process a sample that contain an extracellular vesicle contaminated with host cell DNA. In one such embodiment, the extracellular vesicles are exosomes contaminated with host cell DNA.

In one embodiment of the method of the invention, a sample may previously have been partially purified, including by methods that have the effect of reducing the content of host cell DNA.

Anionic metal affinity substrates suitable to practice the method of the invention include immobilized amino-carboxylic acids. In one embodiment, an immobilized amino-carboxylic acid may be a dicarboxylic acid such as iminodiacetic acid (IDA). In another embodiment, the amino-carboxylic acid may be an immobilized tricarboxylic acids such as nitriloacetic acid (NTA). In another embodiment, a mixture of IDA and NTA substrates may be employed. Anionic metal affinity substrates are available commercially in a variety of physical forms and may be synthesized in any format desired. They may be in the form of particles, insoluble nanofilaments, porous membranes, monoliths, hydrogels, depth filtration media, soluble polymer media, or other formats. In many cases, such substrates are available in the form of a flow-through chromatography device to facilitate their use.

In some embodiments, the choice of anionic metal affinity ligand can contribute to non-retention of the desired protein, virus, or extracellular vesicle product. In some such embodiments, NTA may be preferred over IDA in some embodiments because NTA carries three negative charges where IDA carries only two. Complexes of divalent metal cations with IDA will produce a net charge of zero by the ligand-metal complex but complexes of divalent metal cations with NTA will produce a net charge of minus one (1−), which may tend to discourage binding of the desired product. Complexes of trivalent metal cations with IDA will produce a net charge of plus one (1+) by the ligand-metal complex, which may endow the complex with anion exchange characteristics that favor binding by the desired protein, virus, or extracellular vesicle. Complexes of trivalent metal cations with NTA will produce a net charge of zero, which will not endow the complex with significant anion exchange capability. It will be advisable to evaluate both IDA and NTA for routine application of the method.

It will be understood by persons of experience in the art that non-lipid-enveloped protein-capsid viruses tend to be robust and often tolerate pH 9 over a wide range of salt concentrations. This will make it a simple matter to conduct either or both steps of the method of the invention at a pH of about 9. It will be equally understood that lipid-enveloped viruses, virus-like particles, bacteriophages, and extracellular vesicles are more labile and may require moderation of pH to maintain product stability. In one such embodiment, the metal affinity step may be conducted at a pH of about 8 and a sodium chloride concentration of about 250 mM. Less tolerant species may require reduction to a pH slightly above neutral and a salt concentration close to 100 mM. More robust species may tolerate a pH of 8.75 and a concentration of salt up to 375 mM or more. As a general matter, the lowest concentration of salt required to prevent product binding of the target product during the metal affinity step will be advantageous because it will minimize the degree to which the processed sample must be diluted to bind to the anion exchanger in the final step of the method. Also, in general, the nearer the pH is to neutral, the more likely it will be tolerated by labile products such as those with lipid membranes.

Buffer pH may be in the range of pH 4.0 to pH 10.0, or 5.0 to 9.5, or 6.0 to 9.0, or 6.5 to 8.5, or 7.0 to 8.0, or 6.5 to 7.5, or a different or narrower range, according to the stability requirements of the desired protein, virus, or extracellular vesicle. It will be recognized by persons of knowledge in the art that some buffering agents may interact with metals [10] and may be exploited to modulate the performance of the method of the invention.

In some embodiments, salts which is not forming a chemical complex with the metal-loaded anionic metal affinity substrate (non-chelating salts) may be present at a concentration in the range of 0.1 mM to 1.0 M, or 50 mM to 750 mM, or 100 mM to 500 mM, or 125 mM to 250 mM. In some embodiments, the presence of salt may help to stabilize the virus particles or extracellular vesicles. In some embodiments, exposure of the extracellular vesicles or lipid-enveloped viruses to salt concentrations greater than 500 mM should be brief to minimize damage to the product. In some embodiments, the metal affinity step of the invention will bind chromatin even at salt concentrations of 1 M, or 2 M, or 3 M, or 4 M, or 5 M, or in saturating concentrations of non-chelating salts. It will be recognized that such high concentrations will be seldom or never beneficial to the overall practice of the method of the invention, and especially not when the desired product is labile to such conditions, but such conditions will still support selective removal of chromatin.

In some embodiments, the species of salt which is not forming a chemical complex with the metal-loaded anionic metal affinity substrate (non-chelating salt) employed to conserve stability of the product may be an inorganic salt such as sodium chloride, or potassium chloride, or sodium acetate, or potassium acetate, or another salt.

In closely related embodiments, the non-chelating salt may be an organic salt, such as arginine-HCl, lysine-HCl, or a salt based on an imidazolium, histidyl, or histaminyl cation.

In another closely related embodiment, the non-chelating salt may be a chaotropic salt comprising a guanidinium cation or a thiocyanate anion, or both, or other chaotropic ions. It will be recognized by persons of knowledge in the art however, that the use of such salts will be restricted to proteins and protein-protein capsid viruses since such salts are likely to damage products that possess a lipid membrane.

Anions with strong capacity to bind metal cations will tend to remove the metals bound to the anionic solid phase ligand and will compromise the ability of the anionic metal affinity substrate to bind chromatin. Anions known to have strong metal-binding ability include citrates, phosphates, pyrophosphates, ethylenediaminetetraacetic acid (EDTA), ethylene glycol-bis(β-aminoethyl ether)-N,N,N′,N′-tetraacetic acid also known as egtazic acid (EGTA), aspartic acid, glutamic acid, and glutamine.

Multivalent metal cations suitable to practice the method particularly include ferric iron and manganese. Cupric copper, zinc, magnesium, calcium, and barium may also be employed. Heavy metal ions such as nickel and cobalt, among others, also mediate DNA reduction but their use is discouraged by their toxicity.

In some embodiments, the choice of metal ion can also contribute to non-retention of the desired virus or extracellular vesicle product. Metal ions with notably high affinity for phosphate residues will tend to bind all phosphorylated species more strongly than metal ions with weaker phosphate affinity. Metal ions with high affinity for phosphate residues particularly include ferric iron and manganese. Experimental data indicate that metals such as calcium and magnesium have lower affinity for phosphate. Metals such as cupric copper mediate intermediate affinity for phosphate groups. In embodiments where the desired protein, virus, or vesicle species exhibits inherently low affinity for metal ions bound to an anionic metal affinity solid phase, it may be advantageous to use iron or manganese for the purpose of maximizing chromatin binding. In embodiments where the desired protein, virus, or vesicle species is highly phosphorylated, it may be beneficial to employ copper, calcium, or magnesium for the purpose of maximizing recovery of the desired product.

Many types of anion exchangers are commercially available worldwide. In one such embodiment, the anion exchanger is a quaternary amine anion exchanger, also known as a strong anion exchanger. In another such embodiment, the anion exchanger is a tertiary anion exchanger, also known as a weak anion exchanger. Anion exchangers may also employ primary amino groups, secondary amino groups, and combinations of primary, secondary, tertiary, and quaternary amino groups. One such material is N,N-Bis(2-aminoethyl)-1,2-ethanediamine more commonly referred to as TREN. Other anion exchangers of mixed composition employ ligands consisting of polyallylamine, polyethyleneimine, and ethylenediamine, among others. Anion exchangers suitable to practice the method are also understood to include positively charged amine derivatives that include additional residues to confer excess hydrophobicity, hydrogen bonding, or both. Anion exchangers including additional residues to confer excess hydrophobicity and/or hydrogen bonding are commonly referred to as multimodal or mixed-mode exchangers. Throughout this specification, all of the foregoing materials are referred to as anion exchangers. All of them are commercially available worldwide in a variety of physical forms, including particles, insoluble nanofilaments, porous membranes, monoliths, hydrogels, depth filtration media, or other formats. In many cases they are available in the form of a flow-through chromatography or filtration device to facilitate their use. In one embodiment, the metal affinity substrate and the anion exchanger are both in the form of chromatography devices, plumbed in sequence with the metal affinity device first. In one such embodiment they are equilibrated in tandem, loaded in tandem, washed in tandem, eluted in tandem, and cleaned in tandem. In another such embodiment, they are equilibrated in tandem, loaded in tandem, washed in tandem, then the metal affinity is removed from the flow stream and the anion exchanger is eluted independently. It will be recognized by persons of knowledge in the art that plumbing devices in sequence can be attractive for industrial users because it substantially reduces the amount of water, buffers and salts, buffer preparation, process time, equipment, and staffing required to process a desired product. All of these benefits contribute to higher facility capacity, which ultimately translates to higher productivity at lower expense.

In some embodiments, the metal affinity-treated sample may be applied to the anion exchanger without concern for residual free metal ions in the sample since such metal ions, being positively charged, will be repelled by the surface of the anion exchanger and eliminated during sample application. In other embodiments, metal ions may be deliberately added to the sample and the anion exchange buffers. In such embodiments, their presence may modify the surface charge or surface topography of viruses or vesicles in ways that are beneficial. In one such embodiment, the presence of calcium and or magnesium ions in the buffers contributes to improved separation of empty AAV capsids from full AAV capsids.

In some embodiments, anion exchange chromatography is performed directly following the metal affinity step. In one embodiment where the metal affinity chromatography step and the anion exchange chromatography step are performed in uninterrupted sequence, both media are used in the form of a chromatography device or a filtration device. In another embodiment where the metal affinity chromatography step and the anion exchange chromatography step are performed in uninterrupted sequence, the metal affinity step is performed by adding an insoluble metal affinity substrate to the sample and permitting it to bind and co-precipitate the DNA, then removed so the DNA-deficient supernatant can be applied to the anion exchanger. In another embodiment where the metal affinity chromatography step and the anion exchange chromatography step are performed in uninterrupted sequence, the metal affinity step is performed by adding soluble polymer substrate bearing the metal affinity ligand to the sample, allowing it to cross-link and precipitate the chromatin, then removing the precipitate by centrifugation and/or filtration to yield a DNA-deficient supernatant to be processed by anion exchange chromatography.

In a related embodiment, performed at a salt concentration up to 400 mM sodium chloride, the metal affinity chromatography substrate may be added to a sample in combination with positively charged particles or polymers, which will co-crosslink to the DNA associated with the metal affinity chromatography media and further contribute to DNA reduction. Elevated salt will meanwhile discourage binding of the desired protein, virus, or vesicle with either the substrate of the invention or the positively charged substrate. As a general matter, the concentration of salt need be no higher than necessary to prevent binding the desired protein, virus, or vesicle to the substrate of the invention or to the positively charged substrate. After treatment, the salt concentration may be reduced, if necessary, to enable the sample to be processed by anion exchange chromatography.

In another closely related embodiment, the method of the invention may be combined with treatment by a fatty acid. In some such embodiments, the fatty acid may be heptanoic acid, or octanoic acid, or nonanoic acid at a concentration in the range of 0.01% to 1.0%, and at a pH in the range of 4 to 6. In some such embodiments, the fatty acid may be present at the same time that particles or polymers bearing the metal-loaded anionic metal affinity substrate is present. In some such embodiments, the fatty acid treatment may be conducted before or after the method of the invention. It will be apparent to persons of knowledge in the art that treatments including fatty acids will be unsuitable for viruses and extracellular vesicles with lipid membranes.

In another closely related embodiment, the method of the invention may be combined with treatment with allantoin. In some such embodiments, the allantoin may be present in an amount ranging from 2% to 10%. It will be apparent to persons of knowledge in the art that treatments including allantoin will be unsuitable for some viruses and extracellular vesicles.

In another embodiment one or more additional processing steps may be inserted after the metal affinity step and before the anion exchange chromatography step.

In one such embodiment, the sample is processed after metal affinity by biological affinity chromatography. In one such embodiment, the affinity chromatography ligand is a biological ligand specific for one or more serotypes of AAV. In another such embodiment, the affinity ligand is a biological ligand specific for an antibody. In one such embodiment, the affinity ligand is protein A or a variant thereof.

In a related embodiment, the sample is processed by cation exchange chromatography after the metal affinity step, then processed by anion exchange chromatography. In one such embodiment, the cation exchanger is used to capture AAV. In another embodiment, the cation exchanger is used to capture an antibody

In another related embodiment, the sample is processed by hydrophobic interaction chromatography after metal affinity then processed by anion exchange chromatography.

In another related embodiment, the sample is processed by an anionic immobilized metal affinity chromatography after the anionic metal affinity DNA-removal step, then processed by anion exchange chromatography. In one such embodiment, this variation is applied to biomolecules that naturally bear histidine clusters or which have been produced from recombinant gene constructs that cause them to bear an artificial histidine cluster, tail, or tag. In one such embodiment, IgG, which naturally bears a histidine cluster in its hinge region of the desired product. In one such embodiment, the anionic metal affinity ligand for the DNA removal step bears ferric iron and the anionic metal affinity ligand for the subsequent purification step bears nickel. This step binds IgG. The IgG may be eluted by competition with imidazole, reduction of pH, or a combination of both. The eluted IgG is then polished with anion exchange chromatography. In one such embodiment, the anion exchanger is a multimodal anion exchanger. In a variant of the above embodiment, ferric iron is replaced by manganese. In another variant of the above embodiment, nickel is replaced by copper, zinc, or cobalt. In another variant of the above embodiments, the two metal affinity steps are performed with a pair of columns plumbed together, where the first in the sequence is an IDA column loaded with ferric iron and the second is IDA loaded with zinc. In one such embodiment, filtered cell culture harvest containing IgG monoclonal antibodies is passed through both columns, where the first removes DNA and the second captures IgG. The columns are washed, then a buffer is applied to elute IgG from the second column while DNA remains bound to the first until it is later removed with NaOH. In another variation of this approach, the product of interest, instead of being an antibody, is a His-tagged protein, or a His-tagged exosome, or a His-tagged virus particle.

In another related embodiment, the sample is processed by tangential flow filtration after metal affinity and before anion exchange chromatography. Since virus particles and extracellular vesicles represent large complex assemblages, commonly ranging in size from 20 nm to more than 200 nm, many such embodiments will benefit from integrated processing by tangential flow filtration with the largest pores that retain the product of interest. In some such embodiments, this will involve TFF membranes with pore size cutoffs in the range of 200 kDA to 700 kDA, and in some cases very large pore size ratings such as 1 MDa. Such filters permit the elimination of smaller contaminants by their passage through the pores of the membranes. In one such embodiment, metal affinity particles or polymers loaded with magnesium are mixed with a cell culture harvest of lysate at alkaline pH to bind the DNA. Solids are then removed by centrifugation and/or membrane filtration, and the clarified supernatant is concentrated and/or diafiltered to concentrate and/or buffer exchange the sample in preparation for anion exchange chromatography. In a related embodiment where the desired product is an IgG antibody, the pore size cutoff of the membrane may be 30-50 kDa. In one such embodiment, a subsequent anion exchange chromatography step is conducted with a multimodal anion exchanger. In a closely related embodiment where the desired product is an IgM antibody, the pore size cutoff of the membrane may be 30-100 kDa and a subsequent anion exchange chromatography step is conducted with a strong anion exchanger such as a quaternary amine anion exchanger.

In one embodiment, the method of the invention is used to process adeno-associated virus, where the anion exchanger fulfills the additional function of fractionating empty capsids from full capsids. The term full capsids is understood to refer to capsids which contain their intended payload of therapeutic plasmid DNA. The term empty capsids is understood to refer to capsids lacking the complete therapeutic DNA plasmid. In one such embodiment, the anion exchanger is a strong anion exchanger eluted with an increasing salt gradient. In another such embodiment, the anion exchanger is a primary amine anion exchanger eluted with an ascending pH gradient. In another such embodiment, the anion exchanger is mixed amino anion exchanger. In one such embodiment the anion exchanger is TREN.

In one embodiment, the method of the invention is used to process extracellular vesicles, including exosomes. In one such embodiment, the anion exchanger is a strong anion exchanger eluted with an increasing salt gradient. In another such embodiment, the anion exchanger is a tertiary amine (weak) anion exchanger eluted with a salt gradient.

In one embodiment, the method of the invention is used to replace sample treatment with nuclease enzymes to reduce DNA content. In another embodiment, the method of the invention is used to augment the degree of DNA reduction achieved by treatment with nuclease enzymes. In one such embodiment, the metal affinity step of the invention is performed in advance of treatment with nuclease enzymes. In another such embodiment, the method of the invention is performed after treatment with nuclease enzymes, where it provides additional utility by binding the nuclease enzymes by their associated metal ion cofactors. In one such embodiment, the anionic metal affinity ligand is loaded with the same metal ion species used as a cofactor for the nuclease enzyme. In one such embodiment, the metal ion species is magnesium. In another such embodiment, the metal ion species is calcium. In an embodiment where the metal affinity step is performed at the same time the sample is treated with a nuclease enzyme, the metal ion used to load the affinity substrate is different from the metal ion species than the enzyme cofactor. For example, if the enzyme co-factor is magnesium, the metal affinity ligand may be loaded with ferric iron so that the metal affinity substrate will not bind the enzyme during lysis of DNA. In any of the foregoing embodiments, the metal affinity ligand may be one of many affixed covalently to a plurality of soluble polymers. In another such embodiment, the metal affinity ligand may be affixed covalently to a plurality of insoluble solid phase particles.

In one embodiment where the metal-affinity DNA-reduction step is performed with loose particles or soluble polymers bearing ligand-metal complexes, precipitates and co-precipitates may be formed. These solids may be removed before further processing of the supernatant containing the desired virus or vesicle species. In one embodiment they may be removed by membrane filtration, or centrifugation, or a combination of the two. After removal of solids, the sample may be processed by means of tangential flow filtration (TFF). In one such embodiment, TFF is performed using membranes with the largest pore size that retains the virus or vesicles of interest while but allows smaller contaminating species to be eliminated by their passage through the pores.

In one such embodiment, the pore size cutoff rating may be 100 kDa, or 300 kDa, or 500 kDa, or 700 kDa, or 1 MDa, or a larger or intermediate molecular weight cutoff (MWCO). In most or all of the foregoing embodiments, the TFF step particularly enables elimination of histone proteins liberated by lysis of the host cell DNA they were associated with. In any of the foregoing embodiments, the TFF step may also be used to concentrate the sample and/or diafilter the sample into a buffer suitable for performing a chromatography step.

In any of the foregoing embodiments, treatment of a sample by the metal affinity step may remove large aggregates and cell debris to an extent that render the sample more filterable and easier to process by TFF or chromatography. In one such embodiment, TFF may be used to concentrate the sample and diafilter it into conditions for enzymatic digestion by nuclease enzymes to further reduce DNA levels. In one embodiment, a sample treated by metal affinity may be further processed by TFF to remove histone proteins before processing the sample by anion exchange chromatography, or by an intermediate chromatography step prior to anion exchange chromatography.

In some embodiments, secondary additives may be included in product preparations suppress non-specific interactions between the desired product and processing surfaces or to stabilize the desired product. Such additives may include non-ionic or zwitterionic surfactants such as octaglucoside, poloxamer 188, Pluronic F68, CHAPS, or CHAPSO, among others. Such stabilizing compounds may instead or additionally include sugars such as sucrose, sorbitol, xylose, mannitol, or trehalose, among others. Such stabilizing compounds may instead or additionally include amino acids such as betaine, tauro-betaine, arginine, histidine, or lysine, among others. All of these agents are known in biopharmaceutical field because they tend to improve solubility and/or recovery of stable product. In some cases, they also improve fractionation of a desired product from undesired species.

It will be recognized that both steps of the method of the invention have potential to remove other phosphorylated contaminants as a byproduct of removing chromatin. Other phosphorylated contaminants potentially include RNA, endotoxins, phosphoproteins, and phospholipids.

### EXAMPLES

### Example 1

Advance removal of host cell DNA from a preparation of adeno-associated virus (AAV) by anionic metal affinity with magnesium A monolith bearing iminodiacetic acid (IDA) chelating residues was loaded with magnesium and equilibrated to pH 9.0. A sample of cation exchange-purified capsids was equilibrated to the same conditions and loaded onto the column. AAV capsids passed through the column unbound. Host cell DNA bound and was later removed with 1 M NaOH. Results are illustrated in FIG. 2.

### Example 2

Advance Removal of Host Cell DNA from a Preparation of AAV by Anionic Metal Affinity with Magnesium

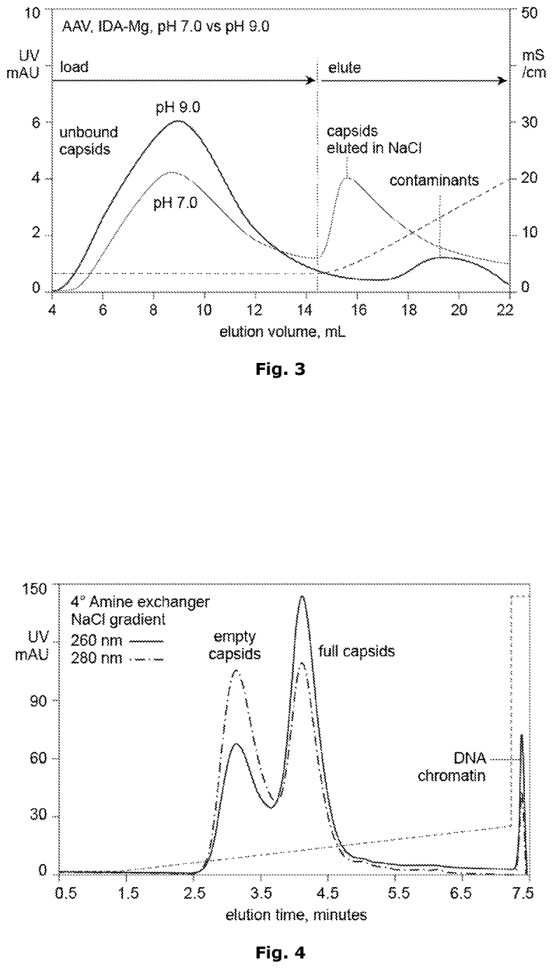

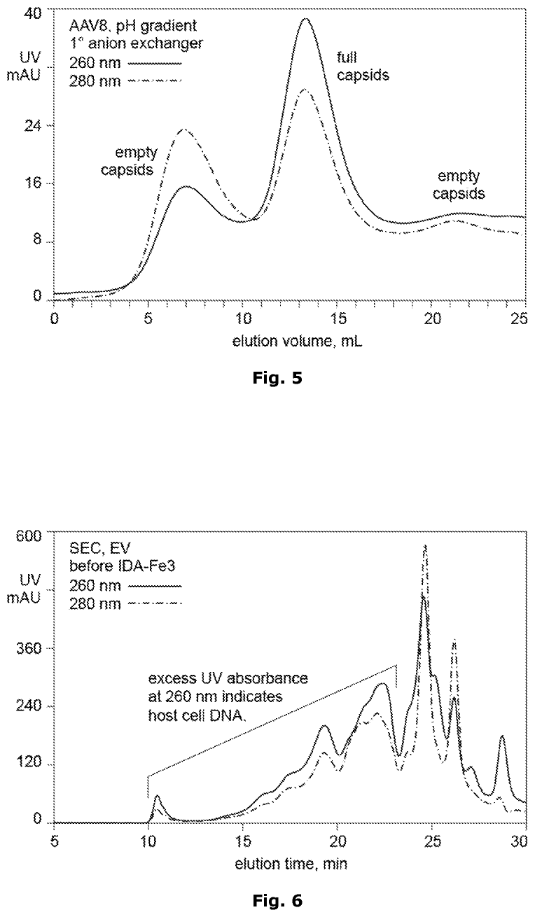

A monolith bearing IDA chelating residues was loaded with magnesium and equilibrated to pH 7.0. A sample of cation exchange-purified capsids was equilibrated to the same conditions and loaded onto the column. AAV capsids were partially bound, along with DNA (FIG. 3. Compare with FIG. 2). Despite partial binding of AAV capsids at pH 7.0, the results show their elution at about 250 mM NaCl. This means inclusion of that amount of salt in the equilibrated sample and column would have prevented their binding. FIG. 4 illustrates separation of empty and full AAV capsids coincident with removal of DNA by anion exchange chromatography with a strong (quaternary amine) anion exchanger eluted with a sodium chloride gradient. FIG. 5 illustrates separation of empty and full AAV capsids coincident with removal of DNA by anion exchange chromatography with a weak (primary amine) anion exchanger eluted with a pH gradient.

### Example 3

Advance Removal of DNA from a Preparation of Extracellular Vesicles

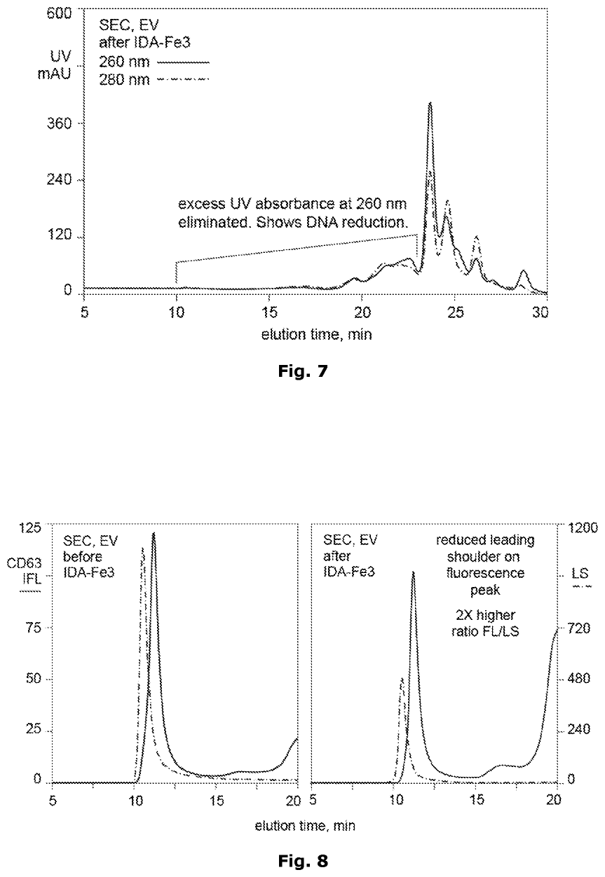

A monolith bearing IDA chelating residues was loaded with ferric iron and equilibrated with 50 mM Hepes, 50 mM NaCl, pH 7.0. A clarified mammalian cell culture harvest was passed through the monolith. FIG. 6 illustrates an analytical size exclusion chromatography (SEC) profile of the sample before it was applied to the IDA-Fe monolith. Note the excess UV absorbance at 260 nm from about 10 minutes to about 23 minutes. This indicates the presence of nucleic acids and corresponds to the zone in which chromatin normally elutes. FIG. 7 illustrates an analytical size exclusion chromatography profile of the sample after it was applied to the IDA-Fe monolith. Note the elimination of excess absorbance at 260 nm and the general reduction of UV signal from 10-23 min. This is consistent with chromatin removal. FIG. 8 illustrates results from before and after analytical size exclusion chromatography monitored by Multi-Angle Light Scattering (MALS, LS) and by immunofluorescence (IFL). Light scatter selectively amplifies optical detection of large solutes such as extracellular vesicles, including exosomes, microvesicles, apoptotic bodies, chromatin, and cell debris. Immunofluorescence (IFL) is performed in conjunction with SEC by adding a fluorescently labeled antibody to the sample before chromatography then monitoring the run with a fluorescence detector. It detects only solutes that bear the specific immunological marker to which the antibody is directed. In this experiment, the antibody was directed to CD63, which is a marker known to be characteristic of exosomes. MALS and IFL signal intensity for the “after treatment” sample were adjusted upward by a factor of 5 to compensate for 5-fold sample dilution during earlier processing by the method of the invention. Note that the ratio of IFL to MALS increased as a result of treatment. This indicates that the metal affinity step of the method selectively removes sample components lacking the exosome marker and thereby produces a more enriched exosome fraction. This suggests that a large proportion of the solutes eluting from 10-12 minutes and detected by MALS in FIG. 6 were associated with chromatin. FIG. 9 illustrates processing of a partially purified extracellular vesicle preparation that was loaded onto a strong anion exchanger (quaternary amine) equilibrated to 50 mM Hepes, 50 mM NaCl, pH 7.0, then eluted with a linear gradient to 2 M NaCl before being cleaned with 1 M NaOH. Extracellular vesicles mostly elute in less than 1 M NaCl. Chromatin mostly requires NaOH for elution.

### Example 4

Advance Removal of Host DNA from a Preparation of Bacteriophage T4

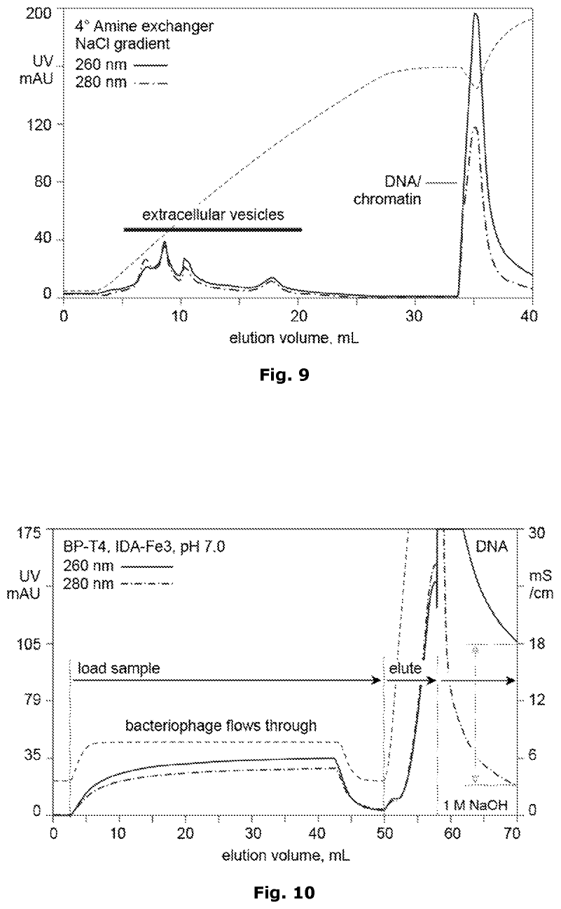

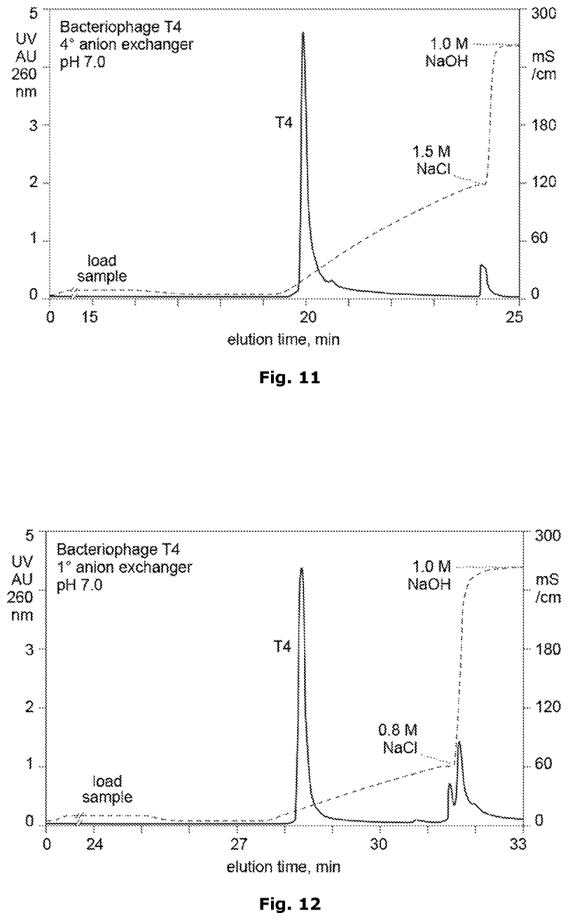

A monolith bearing IDA chelating residues was loaded with ferric iron and equilibrated to pH 7.0. A filtered cell culture harvest was run through the monolith to remove DNA. FIG. 10 illustrates the elution profile. The bacteriophage flowed through the monolith. Some contaminants were bound and eluted with NaCl. DNA was later removed by 1 M NaOH. FIG. 11 illustrates polishing purification by anion exchange chromatography on a strong anion exchanger (quaternary amine) eluted with a sodium chloride gradient at pH 7. FIG. 12 illustrates polishing purification by anion exchange chromatography on a weak anion exchanger (primary amine) eluted with a sodium chloride gradient at pH 7.

### Example 5

Removal of Host DNA from a Preparation Containing an IgG Monoclonal Antibody

An IDA monolith is loaded with ferric iron and excess iron is washed away with 1 M NaCl. The IDA-Fe substrate is washed with water to remove excess salt. Filtered cell culture harvest containing IgG monoclonal antibodies is passed through the monolith under roughly physiological conditions. The term physiological conditions is understood to include a pH of about 6.5 to 7.5 and a salt concentration corresponding to a conductivity of 50-200 mS/cm. The antibody flows through. Chromatin is bound. The monolith is rinsed to recover all of the antibody. The antibody is then processed by multimodal anion exchange chromatography to further reduce DNA content.

### Example 6

### Purification of an IgG Monoclonal Antibody

The method of Example 5 is repeated except inserting a TFF step after metal affinity removal of chromatin. TFF is performed with a membrane with a molecular weight cutoff (MWCO) of 30 kDA to retain the IgG while the content of lower molecular weight proteins and low molecular weight contaminants is reduced before the anion exchange chromatography step. In a variation of this process, metal affinity removal of DNA may be performed by treating the harvest in a bulk format with IDA-Fe particles instead of a flow-through chromatography device as described in Example 5.

### Example 7

Removal of Host DNA from a Preparation Containing an IgM Monoclonal Antibody

An IDA monolith is loaded with ferric iron and excess iron is washed away with 1 M NaCl. The IDA-Fe substrate is washed with water to remove excess salt. Filtered cell culture harvest containing IgM monoclonal antibodies is passed through the monolith under roughly physiological conditions. The antibody flows through. Chromatin is bound. The monolith is rinsed to recover all of the antibody. The antibody is then processed with a strong anion exchanger eluted with a salt gradient to further reduce DNA content.

### Example 8

### Purification of an IgM Monoclonal Antibody

The method of Example 7 is repeated except inserting a TFF step after metal affinity removal of chromatin. TFF is performed with a membrane with a MWCO of 100 kDA to retain the IgM while the content of lower molecular weight proteins and low molecular weight contaminants is reduced before the anion exchange chromatography step. In a variation of this process, metal affinity removal of DNA may be performed by treating the harvest in a bulk format with IDA-Fe particles instead of a flow-through chromatography device as described in Example 7.

### LIST OF REFERENCES

All references cited herein are incorporated by reference to the full extent to which the incorporation is not inconsistent with the express teachings herein.
[1] R Nian, W Zhang, L Tan, J Lee, X Bi, Y S Yang, H T Gan, P Gagnon, Advance chromatin extraction improves capture performance of protein A affinity chromatography, J Chromatogr A 1431 (2016) 1-7.
[2]. R Nian, P Gagnon, Advance chromatin extraction enhances performance and productivity of cation exchange chromatography-based capture of immunoglobulin G monoclonal antibodies, J Chromatogr A 1453 (2016) 54-61.
[3] P Gagnon, R Nian, L Tan, J Cheong, V Yeo, Y S Yang, H T Gan, Chromatin-mediated depression of fractionation performance on electronegative multimodal chromatography media, its prevention, and ramification for purification of immunoglobulin G, J Chromatogr A 1374 (2014) 145-155.
[4] T McNerney, A Thomas, A Senczuk, K Petty, X Zhao, R Piper, J Carvalho, M Hammond, S Sawant, J Bussiere, pDADMAC flocculation of Chinese hamster ovary cells: enabling a centrifuge-less harvest process for monoclonal antibodies, mAbs 7 (2015) 413-427.
[5] J Murphy, D Jewell, K White, G Fox, R Wilson, Nucleic acid separations utilizing immobilized metal affinity chromatography, Biotechnol Progr 19 (2003) 982-986.
[6] T Cano, J Murphy, G Fox, R Wilson, Separation of genomic DNA from plasmid DNA by selective renaturation with immobilized metal affinity capture, Biotechnol Progr 21 (2005) 1472-1477.
[7] M Lock, Luc Vandenberghe, J Wilson, Scalable production method for AAV, U.S. Pat. No. 9,198,984B2, Adjusted expiration Aug. 31, 2028.
[8] M Lock, Luc Vandenberghe, J Wilson, Scalable production method for AAV, US Patent US20160040137A1, Anticipated expiration Apr. 4, 2027.
[9] M Lock, M Alvira, Scalable purification method for AAV9, World patent application WO2017160360A9, Priority date Dec. 11, 2015.
[10] C Ferreira, I Pinto, E Soares, H Soares, (Un)suitability of the use of pH buffers in biological, biochemical, and environmental studies and their interaction with metal ions—a review, RSC Adv 5 (2015) 30989-31003.

1. A method for removal of host cell DNA from a sample containing a desired species of a protein, a virus, or an extracellular vesicle comprising the steps of:
Loading a substrate bearing an anionic metal affinity ligand with a metal ion,
Equilibrating the substrate with a buffer having a pH in the range of pH 4 to pH 10, and a salt concentration in a concentration range up to 1 M which salt is not forming a chemical complex with the anionic metal affinity ligand,
Contacting the sample with the metal-loaded anionic metal affinity substrate,
Separating the substrate from the sample, wherein the sample has a reduced content of host cell DNA.

2. The method of claim 1 wherein the anionic metal affinity ligand is selected from the group consisting of amino-dicarboxylic acids and amino-tricarboxylic acids.

3. The method of claim 2 wherein the anionic metal affinity ligand is iminodiacetic acid (IDA) or nitriloacetic acid (NTA).

4. The method of claim 1 wherein the substrate bearing an anionic metal affinity ligand is in the form of particles, nanofilaments, porous membranes, monoliths, hydrogels, depth filtration media, or soluble polymer media.

5. The method of claim 4 wherein the substrate bearing an anionic metal affinity ligand is in the form of a flow-through chromatography device.

6. The method claim 1 wherein equilibrating the substrate is performed by means of a buffer having a pH in the range of pH 7.0 to 9.5, or 8.0 to 9.0.

7. The method of claim 1 wherein equilibrating the substrate is performed by means of a buffer having a concentration of the salt in the range of up to 1 M.

8. The method of claim 1 wherein the buffer used for equilibrating the substrate provides salt conditions that prevent binding of a virus or extracellular vesicle but permit binding of DNA and are adjusted with the salt selected from the group consisting of an inorganic salt, an organic salt, and a chaotropic salt, and combinations thereof.

9. The method of claim 1 wherein the metal-loaded anionic metal affinity substrate is loaded with metal ions having at least two positive charges.

10. The method of claim 1 wherein
the sample containing the desired species is selected from the group consisting of a cell harvest, cell lysate, and a partially purified preparation thereof and
the desired species is selected from the group consisting of non-lipid-enveloped protein capsid virus particles, lipid-enveloped virus, virus-like particles, bacteriophages, extracellular vesicles, proteins, and combinations thereof.

11. The method of claim 10 wherein the AAV capsid is selected from the group consisting of AAV serotypes AAV1, AAV2, AAV3, AAV4, AAV5, AAV6, AAV7, AAV8, AAV9, AAV10, AAV11, a recombinant hybrid serotype like AAV2/8, or AAV2/9, a synthetic recombinant serotype and combinations thereof.

12. The method of claim 1 wherein the sample is processed by biological affinity chromatography after the metal affinity step of claim 1, cation exchange after the metal affinity step of claim 1, hydrophobic interaction chromatography after the metal affinity step of claim 1, tangential flow filtration after the metal affinity step of claim 1 or combinations thereof.

13. The method of claim 12 wherein the tangential flow filtration is using a membrane with pore size cutoffs in the range of up to 1 MDa.

14. The method of claim 1 wherein the sample having a reduced content of DNA is further processed by anion exchange chromatography.

15. The method of claim 1 wherein the sample containing the desired species is a cell harvest or cell lysate.

16. The method of claim 15 wherein the sample did not undergo any chromatographic step prior to the step of contacting the sample with the metal-loaded anionic metal affinity substrate.

17-19. (canceled)

20. The method of claim 1 wherein the sample has alkaline conditions.

21. The method of claim 1 wherein equilibrating the substrate is performed by means of a buffer having a concentration of the salt in the range of 125 mM to 250 mM.

22. The method of claim 8 wherein
the inorganic salt is selected from the group consisting of sodium chloride, potassium chloride, sodium acetate, and potassium acetate,
the organic salt is selected from the group consisting of arginine-HCl, lysine-HCl, a salt based on an imidazolium, histidyl, and histaminyl cation, and
the chaotropic salt is selected from a guanidinium cation and a thiocyanate anion.

23. The method of claim 9 wherein the metal ions having at least two positive charges are selected from the group consisting of iron(II), manganese(II), calcium(II), magnesium(II), copper(II), zinc(II), barium(II), nickel(II), cobalt(II), and combinations thereof.
